(19) United States
(12) Patent Application Publication
(10) Pub. No.: US 2013/0023723 A1
(43) Pub. Date: Jan. 24, 2013

(54) SYSTEMS AND METHODS FOR TREATMENT OF FECAL INCONTINENCE

(71) Applicants: Ravinder Mittal, La Jolla, CA (US); Matthew T. Yurek, San Diego, CA (US); Bryan Knodel, Flagstaff, AZ (US)

(72) Inventors: Ravinder Mittal, La Jolla, CA (US); Matthew T. Yurek, San Diego, CA (US); Bryan Knodel, Flagstaff, AZ (US)

(73) Assignee: BioStretch Med, Inc., La Jolla, CA (US)

(21) Appl. No.: 13/475,628

(22) Filed: May 18, 2012

Related U.S. Application Data
(60) Provisional application No. 61/488,685, filed on May 20, 2011.

(51) Int. Cl. A61F 2/02

(52) U.S. Cl. 600/30

(57) ABSTRACT

Disclosed herein are systems and methods of performing a sphincter repair in a patient. The methods can include inserting a stabilization and an anchor deployment tool into the patient, positioning the stabilization tool to access a muscle associated with a sphincter, stabilizing tissue with the stabilization tool, the tissue including or in communication with the muscles associated with the sphincter, advancing the anchor deployment tool into the tissue cavity, advancing the plurality of tissue anchors out of the anchor deployment tool and transmurally through the tissue; tensioning the anchors to shorten the muscle associated with the sphincter, and withdrawing the stabilization tool from the patient.

### BACKGROUND

This application claims the benefit under 35 U.S.C.§119(e) of U.S. Prov. App. No. 61/488,685 filed on May 20, 2011, the disclosure of which is hereby incorporated by reference in its entirety.

### BACKGROUND

1. Field of the Invention

The invention generally relates to systems and methods for treating sphincter dysfunction, including fecal and urinary incontinence.

2. SUMMARY

In some embodiments, disclosed herein are methods of performing a sphincter repair in a patient. The methods can include inserting a stabilization and an anchor deployment tool into the patient; the anchor deployment tool housing a plurality of tissue anchors connected to tension elements housed within the anchor deployment tool; positioning the stabilization tool to access a muscle associated with a sphincter; stabilizing tissue with the stabilization tool, the tissue including or in communication with the muscles associated with the sphincter; advancing the anchor deployment tool into the tissue cavity; advancing the plurality of tissue anchors out of the anchor deployment tool and transmurally through the tissue; tensioning the anchors to shorten the muscle associated with the sphincter; and withdrawing the stabilization tool from the patient. The stabilization tool can be inserted, for example, in the anal canal, vagina, or perineal space. The muscle can be, for example, the internal anal sphincter, the external anal sphincter, and/or the levator ani including the puborectalis muscle. The anchors can include a proximal end, a distal end, an elongate body, and a central lumen. The central lumen can have a first inner diameter at the distal end that is at least 10% smaller than a second inner diameter at the proximal end. The proximal end can have a surface that is angled by between about 5 degrees and about 75 degrees with respect to a longitudinal axis of the anchor. The anchor can have an axial length of between about 4 mm and about 20 mm. Advancing the plurality of tissue anchors out of the anchor deployment tool can include advancing the tissue anchors at an angle of between about 15 degrees and about 75 degrees from a longitudinal axis of the anchor deployment tool. The sphincter could include the anal sphincter. Tensioning the anchors could include applying a force to the tension elements connected to the anchors.

In some embodiments, disclosed is a method of treating fecal incontinence in a patient. The method could include inserting a tool to stabilize the tissue and an anchor deployment tool into the patient; the anchor deployment tool housing a plurality of tissue anchors connected to tension elements housed within anchor deployment tool into a patient; positioning the stabilization tool to access at least one muscle selected from the group consisting of the internal anal sphincter muscle, the external anal sphincter muscle, and the puborectalis muscle; stabilizing tissue with the stabilization tool to create a tissue fold, the tissue in communication with or comprising the sphincter muscles; creating a pocket within the tissue space; advancing the anchor deployment tool into the cavity; advancing the plurality of tissue anchors out of the anchor deployment tool and transmurally through the tissue; tensioning the anchors to shorten the muscle associated with the sphincter; and withdrawing the stabilization tool from the patient.

Also disclosed herein in some embodiments are systems for treating incontinence, including an insertion tool for creating a plication to draw the tissue to be plicated into a suction cavity of the insertion tool, and assist in anchor deployment into the tissue. The suction cavity includes one, two, or more suction ports in communication with a source of vacuum connected to a port located more proximally on the tool. The insertion tool has a proximal end, a proximal handle, and a distal end. The insertion tool has a lumen at the proximal end configured to slidably house the anchor delivery tool therethrough, and coaxial or parallel to the longitudinal axis of the anchor delivery tool. The distal end of the anchor delivery tool when inserted into the insertion tool enters the suction cavity at port. In some embodiments, the insertion tool has a diameter of between about 15 mm and about 35 mm, such as between about 20 mm and about 30 mm, and about 25 mm.

### BRIEF DESCRIPTION OF THE DRAWINGS

FIG. 1 is a view of various structures of the female anatomy in the perineal region.

FIG. 2 is a cut-away view illustrating the anus, rectum, levator ani muscle including the puborectalis, and approximate locations of the internal anal sphincter and the external anal sphincter.

FIG. 3 schematically illustrates the anus and the puborectalis muscle, having bilateral internal plications, according to some embodiments of the invention.

FIG. 4 is similar to the schematic of FIG. 3, except showing bilateral external plications, according to some embodiments of the invention.

FIG. 5 schematically illustrates the internal and external anal sphincters, having external plications, according to some embodiments of the invention.

FIG. 6 is similar to the schematic in FIG. 5, except showing internal plications.

FIG. 7 illustrates one embodiment of a plication tool in a perspective view; FIG. 8 is a side view of the embodiment shown in FIG. 7.

FIGS. 9-10 illustrate cross-sectional views through line A-A of FIG. 8. FIG. 9 shows the system prior to tensioning the tissue and FIG. 10 shows the system after the tissue has been tensioned.

FIG. 11 illustrates a step in plicating tissue using the stapling plication tool, according to some embodiments of the invention.

FIG. 12 is a cross-sectional schematic view illustrating insertion of an instrument, into the perineal space, according to some embodiments of the invention.

FIG. 13 illustrates an anchor delivery tool having a proximal end, distal end, and elongate body, according to some embodiments of the invention.

FIG. 14 is an exploded view illustrating additional features of the anchor delivery tool, according to some embodiments of the invention.

FIG. 15 illustrates a close-up view of an anchor, according to some embodiments of the invention.

FIG. 16 schematically illustrates the distal portion of the needle shaft within the central lumen of the anchor, according to some embodiments of the invention.

FIG. 17 illustrates the interrelationship of a plurality of anchors crossing over each other, as described and illustrated in connection with FIG. 14 above.

FIG. 18 illustrates anchors within the pathways of the housing and ready for delivery, according to some embodiments of the invention.

FIG. 19 illustrates an insertion tool, for creating a plication to draw the tissue to be plicated into a suction cavity of the insertion tool, and assist in anchor deployment into the tissue, according to some embodiments of the invention.

FIG. 20 illustrates the distal end of the anchor delivery system advanced distally into the suction cavity, according to some embodiments of the invention.

As illustrated in FIG. 21, paired anchors can then be advanced distally from the pathways of the housing of the anchor delivery system through a tissue path created by needles and deployed through one or more of the external anal sphincter, internal anal sphincter, or levator ani muscles, including the puborectalis muscle.

FIG. 22 illustrates the insertion tool in the anal canal with suction applied to the tissues and the distal end of the anchor delivery system, according to some embodiments of the invention.

FIG. 23 is a cross section of FIG. 22 showing the insertion tool in the anal canal, the anchor delivery system in the submucosal plane and two anchors delivered through one or more of the external anal sphincter, internal anal sphincter, or levator ani muscles, including the puborectalis muscle.

FIG. 24 illustrates the insertion tool in the anal canal, the anchor delivery system in the submucosal plane and two anchors delivered through levator ani muscles, including the puborectalis muscle, according to some embodiments of the invention.

FIG. 25 is a schematic view of the anal canal showing an external plication of the internal and external anal sphincters, according to some embodiments of the invention.

### DETAILED DESCRIPTION

Sphincter muscles are generally arcuate-shaped muscles that can contract to close off a body passage or opening. Sphincter muscles are present in several anatomical locations, and include, for example, the upper and lower esophageal sphincter, pyloric sphincter (stomach), papillary sphincter (iris), orbicularis oculi (extraocular muscle), orbicularis oris (mouth), sphincter of Oddi (duodenum), ileocecal sphincter, smooth and skeletal muscle uretheral sphincter, and internal and external anal sphincters. In many cases, there are more than one muscle group that control the sphincter opening; e.g. the anal opening is controlled by the internal anal sphincter, external anal sphincter and puborectalis muscle. The puborectalis muscle controls the opening of not only the anal sphincter but also the vagina and urethral sphincter. Sphincter muscle(s) can surround a portion of tissue comprising a passage or opening of the body, such as a canal or tract, and control passage of bodily fluid by contracting. Passage of bodily fluids into or out of the associated passage or openings cannot be sufficiently controlled if the sphincter muscle suffers from dysfunction or damage. For example, the most common cause of anal sphincter and puborectalis muscle (part of the levator ani) injury is obstetric trauma during vaginal delivery. The risk of sphincter injury is increased by a laceration that extends into the rectum (a fourth-degree tear), infection of an episiotomy or laceration repair, prolonged labor, and possibly by the use of a midline episiotomy. Sphincter damage also may result from hemorrhoidectomy, sphincterotomy, abscess drainage, or fistulotomy. Patients with incontinence and a suspected sphincter injury can be evaluated with anal manometry, EMG, and endoanal ultrasound, for example. More severe incontinence may require surgical repair.

The anal and puborectalis muscle sphincter can also be damaged by trauma, such as impalement, blast injury, or crush injuries of the pelvis. Because damage to the anal sphincter is not life-threatening, definitive repair of the sphincter is often deferred until other injuries have been repaired and the patient's clinical condition has been stabilized. Isolated sphincter injuries that do not involve the rectum may be repaired primarily. Rectal injury accompanied by sphincter injury can be treated with fecal diversion, distal rectal washout, and drain placement. Significant perineal tissue loss may require extensive debridement and a diverting colostomy. Prolapse of the rectum may also be associated with a weak or damaged anal sphincter/puborectalis muscle and require surgical repair.

The financial cost of fecal incontinence is significant. More than $400 million is spent each year for adult diapers that control urinary and fecal incontinence. Fecal incontinence is the second leading cause of admission to long-term care (e.g., skilled nursing care) facilities in the United States. In younger patients who desire treatment and correction, the costs are surprisingly high. In a 1996 study of 63 patients treated for fecal incontinence secondary to obstetric injuries, the average cost of treatment per patient was $17,166 (Mellgren A. et al. Dis Colon Rectum. July 1999;42(7):857-65).

Surgical repair techniques such as overlapping sphincteroplasty, postanal intersphincteric levatoroplasty, gracilis muscle transposition, electrostimulation, prosthetic sphincter placement, sacral nerve stimulation are not reliably successful and often have unacceptably high associated morbidity. Diverting colostomies can be effective, but are inconvenient and can carry a social stigma for the patient. Absorbent pads provide some relief, but also may carry a social stigma and do not address the underlying condition. Improved, less invasive methods are needed for the treatment of fecal incontinence and other causes of sphincter dysfunction.

Methods and apparatus to treat urinary incontinence include bladder neck fixation to the back of the symphysis pubis, repair of the levator ani muscle, different types of tapes that are placed transvaginally or using other techniques and sacral nerve stimulation devices.

Methods and apparatus for vaginal rejuvenation include various types of surgeries to decrease the diameter of the vagina.

Systems and methods are disclosed herein for the repair of damaged or scarred tissues to repair a sphincter to treat a medical condition, such as fecal incontinence, urinary incontinence and vaginoplasty. In some aspects, a stapling plication tool can be used to plicate the internal anal sphincter, external anal sphincter, or a levator ani muscle, such as the puborectalis muscle. A system involving a tool configured to be inserted into the anal cavity, vagina, or perineal space, for example, having a suction port and an anchor deployment tool can also be utilized to deploy one, two, or more anchors into a structure associated with continence, such as a muscle or muscle group as described above. Utilization of anchors (connected to tension elements such as sutures) can be advantageous in creating plications, in that the anchors provide an increased footprint/effective surface area for retention against the tissue wall compared with suture loops without anchors, which may be more likely to fail due to migration or attachment.

Systems and methods as disclosed herein can also be used or adapted for use in other applications, including treatment of urinary incontinence, vaginoplasty, reflux disease, endoscopic surgery, natural orifice trans-endoscopic surgery, tissue repair (dermal, mucosal, musculature, etc.), cardiovascular and circulatory repair and extraocular movement disorders.

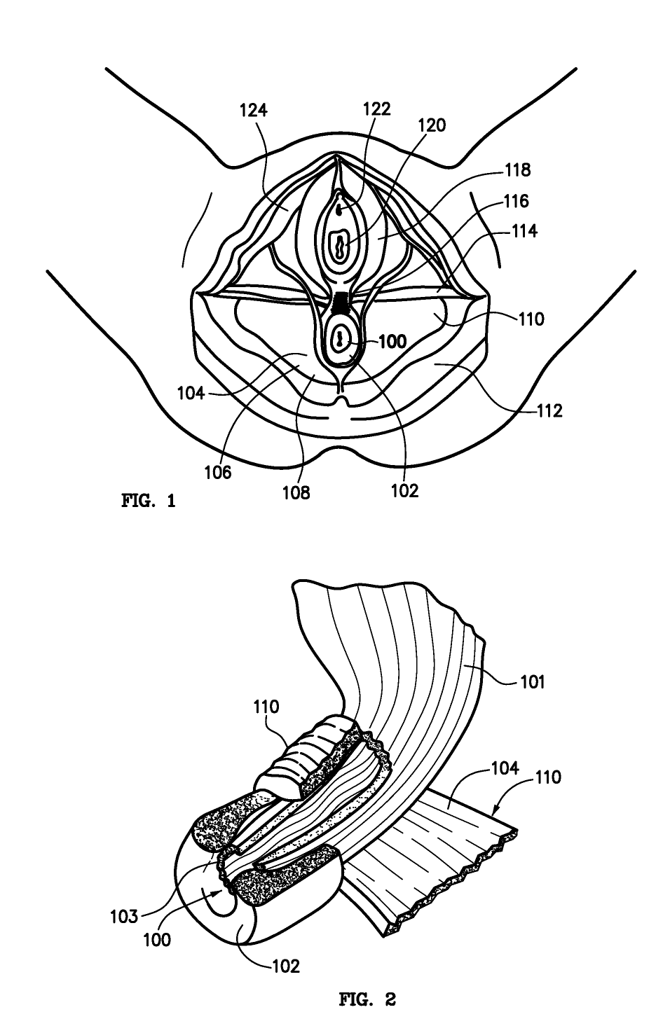

FIG. 1 is a view of various structures of the female anatomy in the perineal region. Illustrated is the anus 100, which is circumscribed by the internal anal sphincter (not shown) and the external anal sphincter 102. The internal anal sphincter is an involuntary muscle innervated by autonomic nerves, while the external sphincter 102 is a voluntary muscle innervated with somatic nerves. The external sphincter 102 has two strata: a subcutaneous and superficial component. The levator ani muscle 110 is also associated with fecal continence and is made up of the puborectalis 104, pubococcygeus 106, and ileococcygeus 108 muscles. The anatomical anal canal extends proximally about 2.5-4 cm from the external anal verge, while the surgical anal canal extends proximally about 4-5 cm from the external anal verge. Also illustrated are the gluteus maximus muscle 112, superficial transverse perineal muscle 114, central perineal tendon 116, bulbospongiosus muscle 118, vagina 120, urethra 122, and ischiocavernosus muscle 124.

FIG. 2 is a cut-away view illustrating the anus 100, rectum 101, levator ani muscle 110 including the puborectalis, and approximate locations of the internal anal sphincter 103 and the external anal sphincter 102.

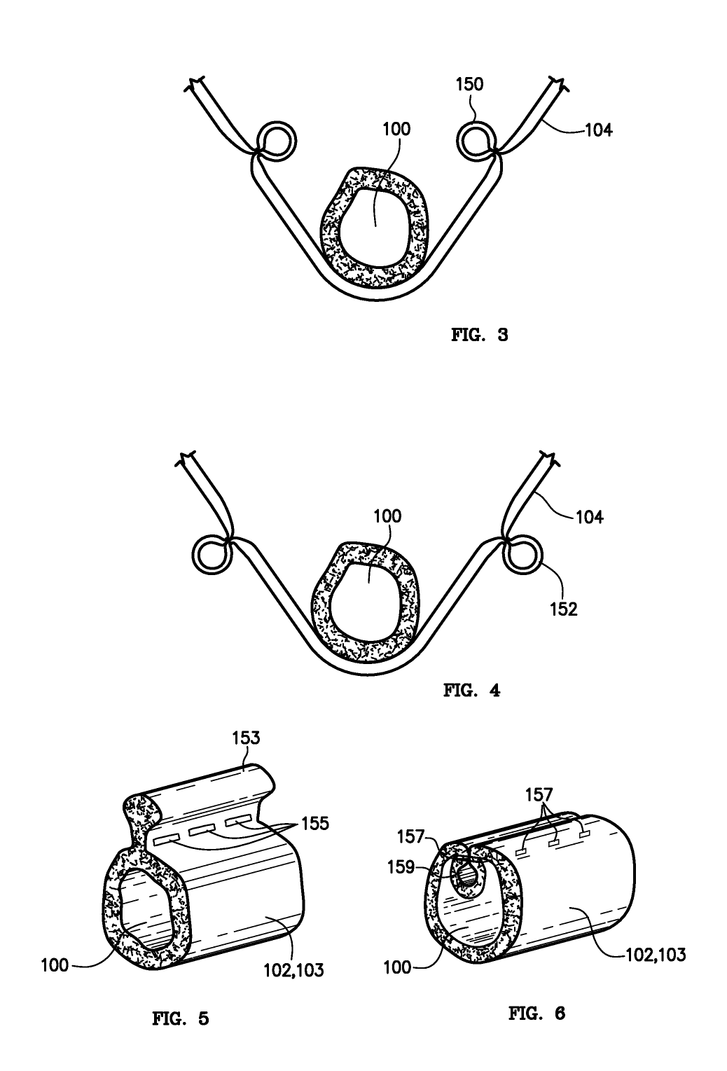

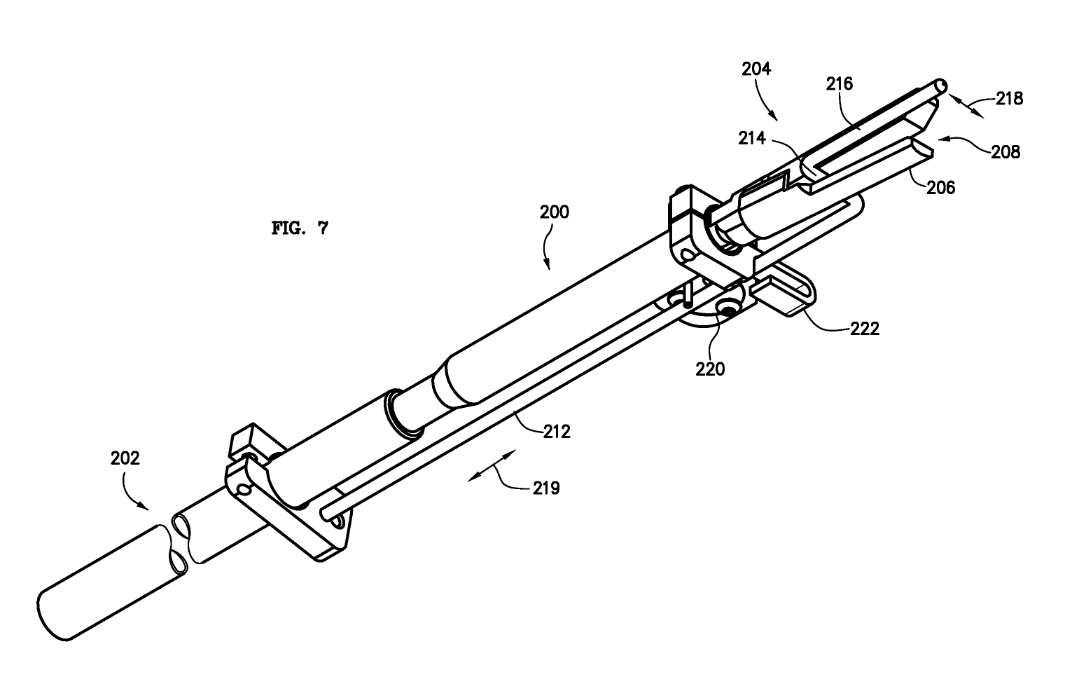

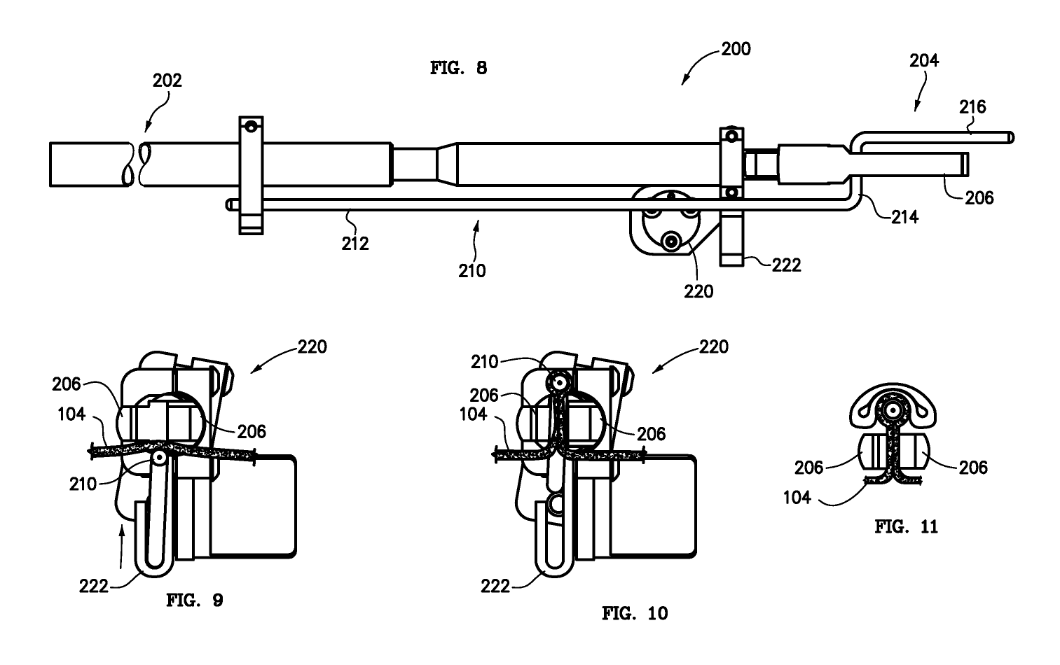

FIG. 3 schematically illustrates the anus 100 and the puborectalis muscle 104, having bilateral internal plications 150 (internal referring to the plication extending generally toward the midline). The plications 150 shorten the puborectalis muscle 104, effectively reducing the pelvic floor hiatus and thus increasing anal canal pressure tension. FIG. 4 is similar to the schematic of FIG. 3, except showing bilateral plications that extend laterally 152 (lateral referring to the plication projecting outside of pelvic hiatus, into the ischio-rectal fossa) rather than the medial plications 150 illustrated in FIG. 3. FIG. 5 illustrates an external anal plication 153 that can be secured using staples 155, anchors, an adhesive or sclerosing agent, or other means as described, for example, elsewhere herein. FIG. 6 illustrates an internal anal plication 159 secured using staples 157 or other means. While primarily described herein with respect to the female anatomy, the systems and methods as described herein can readily be applied to male patients as well. While individual or groups of muscles can be plicated as schematically illustrated, plications can also be created that include a plurality of tissue layers, such as, for example, the serosa, mucosa, and submucosa. Furthermore, the plications can be have different formations, for example, they can overlap, invaginate or project outside of the lumen.

A variety of tools can be utilized to create a plication, which can advantageously increase muscle tension to repair or improve sphincter tone, for example. One embodiment of a stapling plication tool 200 is illustrated in FIG. 7, which is a perspective view, and FIG. 8, which is a side view. FIGS. 9-10 illustrate cross-sectional views through line A-A of FIG. 8. FIG. 9 shows the system prior to tensioning the tissue (e.g., the puborectalis muscle 104) and FIG. 10 shows the system after the tissue has been tensioned in the space between the jaws 208. The tool 200 has a proximal end 202 and a distal end 204. The distal end 204 of the tool 200 includes a pair of movable opposing jaws 206 separated by a space 208. The pair of jaws 206 can be configured to release one, two, or more anchors, such as staples, in between the space 208 when a tissue fold is drawn into the space. The tool 200 also includes a movable bar 210 having a proximal portion 212 that is generally parallel to the longitudinal axis of the tool 200, an intermediate portion 214 that is generally transverse to the longitudinal axis of the tool 200, and a distal portion 216 that is generally parallel to the longitudinal axis of the tool. The bar 210 can be movable in a direction generally transverse to the longitudinal axis of the tool 200 as shown by arrow 218, as well as a direction generally parallel to the longitudinal axis of the tool as shown by arrow 219. Movement of an actuator, such as a rotatable cam 220 operably connected to the bar 210 will move the bar 210 through the space 208, limited and held in position by stop surface 222. After the bar 210 moves through the space 208, opposing jaws 206 can close to deploy an anchor, such as a staple through a fold of tissue (e.g., puborectalis muscle 104) that is moved into the space 208 by movement of the bar 210, as illustrated in FIG. 11.

In some embodiments, the tool 200 can be used to create one, two, or more plications of tissue layers that can include one, two, or more muscles associated with continence of feces, such as the internal anal sphincter, external anal sphincter, and the levator ani, including the puborectalis muscle. For example, to create an internal plication, the distal end 204 of the tool 200 including movable opposing jaws 206 can be inserted into the anus approximately 2-6 cm, such as about 4 cm. The moveable bar 210 can be inserted percutaneously through the perineal skin in a direction substantially parallel to that of the distal end 204 of the tool 200 into the anus, such that at least one muscle associated with continence of feces lies between the bar 210 and the distal end 204 of the tool. The bar 210 could have a sharpened distal tip to facilitate percutaneous insertion, or in other embodiments have a blunt tip. In embodiments where the bar 210 has a blunt tip, a trocar or other sharp-tipped instrument could first create the percutaneous pathway, and then be withdrawn and the bar 210 is then inserted. Following insertion of the bar 210, actuation of the cam 220 as previously described can move the bar 210 in a direction transverse to the longitudinal axis of the tool 200, creating a muscle layer fold, and allowing the movable opposing jaws 206 to staple the fold into a plication.

In other embodiments, the tool 200 can create an external plication by placing the bar 210 into the anus and the distal end 204 of the tool 200 percutaneously. In some embodiments, instead of, or in addition to, a bar 210, the distal end 204 of the tool 200 could include a vacuum port connectable to a vacuum source at or near the proximal end of the tool for creating the plication.

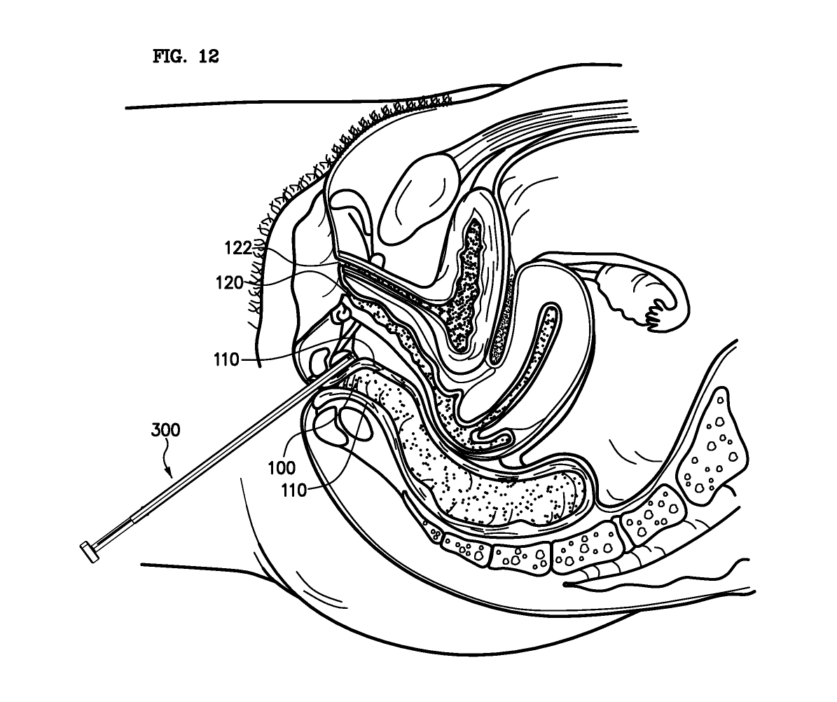

FIG. 12 is a cross-sectional schematic view illustrating insertion of an instrument, such a trocar, the distal end 204 of the stapling tool 200 or the bar 210 described above for example, or an anchor delivery tool 300 as described further below. The instrument can be inserted percutaneously into the perineal space, for deployment of tissue anchors connected to tension elements, which can create a plication if a plurality of tension elements, such as suture segments, are tied to each other. The instrument could also be placed anywhere around the rectum such that the resulting anchors, sutures or staples can tension one or more of the internal anal sphincter, external anal sphincter, and the levator ani, including the puborectalis muscle. Other areas around the rectum include but are not limited to the mucosa, submucosal, intra-costal spaces between muscles and in the fascia or fatty tissues outside of the sphincter muscles.

In some embodiments, plications can be performed using features of clamping and stapling devices as described, for example, in U.S. Pat. Nos. 5,403,326, 5,571,116, and 5,676,674 all to Bolanos et al.; and U.S. Pat. No. 5,403,326 to Harrison et al., all of which are hereby incorporated by reference in their entireties.

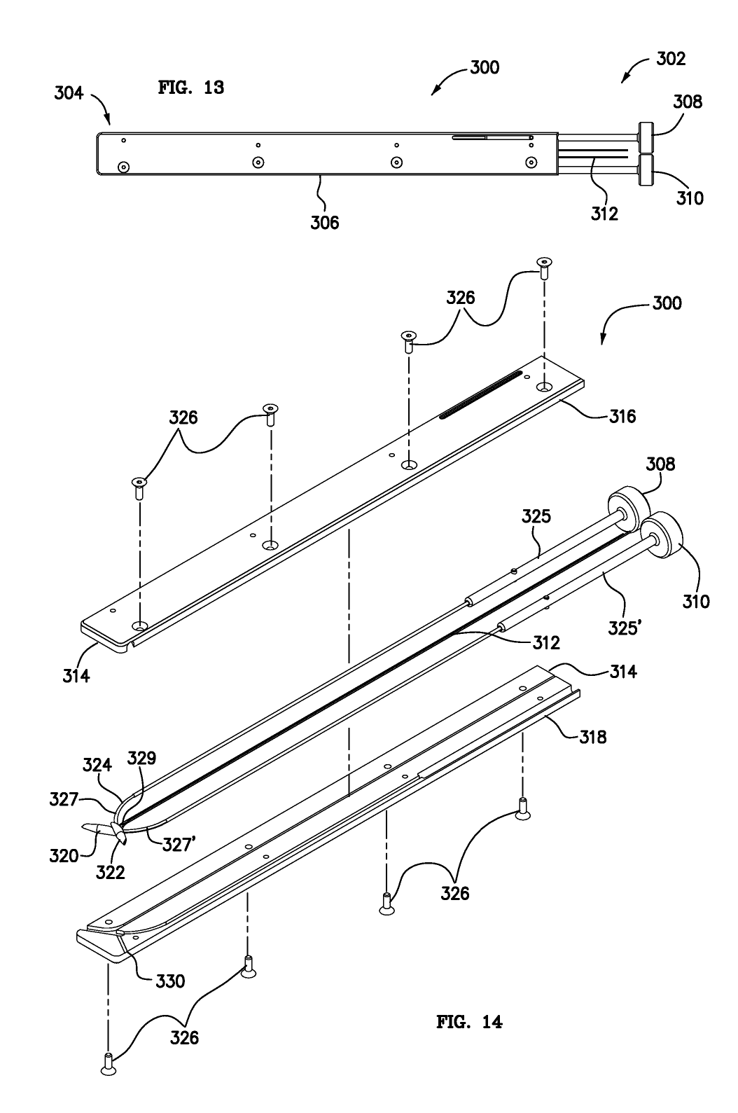

FIG. 13 illustrates an anchor delivery tool 300 having a proximal end 302, distal end 304, and elongate body 306, according to one embodiment of the invention. The anchor delivery tool can be configured to deliver at least one, two, three, or more anchors, each connected to sutures, through tissue, such as one, two, or more muscle layers associated with fecal continence. Tensioning (e.g., by tying, placing a clip or crimping a ferrule) a plurality of sutures together that are each operably connected to an anchor that is delivered through a tissue layer can create a plication, shortening the muscle and treating incontinence. The proximal end 302 of the tool 300 includes handles 308, 310 that can be movable in concert or independently. Also illustrated extending from the proximal end 302 of the tool 300 are tension elements, such as sutures 312. The sutures 312 may be made of any material including resorbable and nonresorbable sutures, and elastic and inelastic sutures.

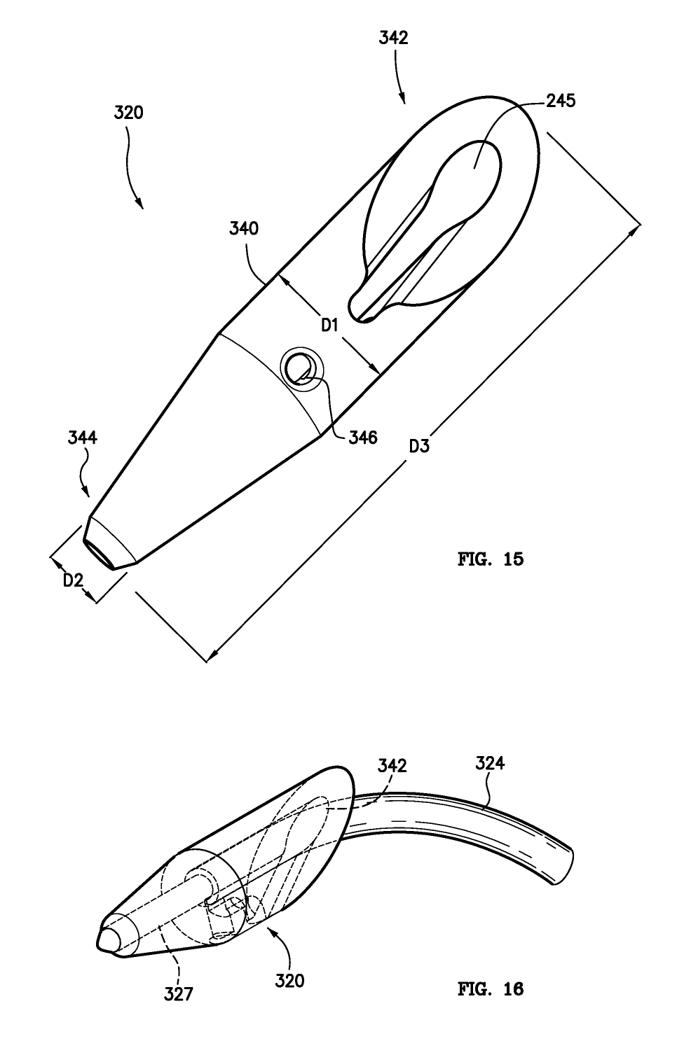

FIG. 14 is an exploded view illustrating additional features of the anchor delivery tool 300. Illustrated are the anchors 320 having a central lumen 322 in which a needle shaft 324 can slide therethrough. Anchors 320 are also operably connected to sutures 312 that run proximally with respect to the anchors for manipulation by a physician. Also illustrated in FIG. 14 is an anchor delivery tool housing 314 including a first portion 316 and a second portion 318, that are connectable via screws 326 as illustrated, although a snap-fit, latch, adhesives, or other means of connecting the components can also be utilized. In some embodiments, the first portion 316 and the second portion 318 of the housing 314 can be integrally formed. The housing 314 could be rectangular-shaped as illustrated, although a tubular or other configuration and/or curved shapes depending on the access orientation for the housing 314 are also possible. The second portion 318 of the housing 314 includes pathways 330 for both the sutures 312 and one, two, or more needle shafts 324 to slide therethrough. As illustrated, the anchor delivery tool 300 can be configured such that the proximal portions 325, 325′ of the two needle shafts 324, 324′ are parallel or substantially parallel to each other. The distal portions 327, 327′ of the needle shafts 324, 324′ can both deflect and cross over each other at point 329. In some embodiments, the paths can be configured such that the distal ends 327, 327′ of the needles 324, 324′ are curvable and exit the pathways at an angle of between about 0 degrees and 180 degrees, between about 30 degrees and 90 degrees, or about 45 degrees depending on the desired insertion trajectory through the tissue wall. Needles 324, 324′ can be made of any desired material, including Nitinol, Elgiloy, or stainless steel. In some embodiments, the anchor delivery tool has a longitudinal axis dimension (length) of between about 10 cm to 20 cm (e.g., about 15 cm), a transverse axis dimension (width) of between about 1 cm and 2 cm (e.g., about 1.4 cm), and a sagittal (thickness) dimension of between about 0.2 cm and about 1 cm (e.g., about 0.5 cm). The distal end of the delivery tool could also be shaped (tapered) to facilitate dissection and separation of tissues as the delivery tool is pushed forward.

FIG. 15 illustrates a close-up view of an anchor 320, according to one embodiment of the invention. Utilization of anchors (connected to tension elements such as sutures) can be easier to deliver and be advantageous in creating plications, in that the anchors provide an increased footprint/effective surface area for retention against the tissue wall compared with suture loops without anchors, which may be more likely to fail due to migration or attachment. The anchor 320 has an elongate body 340, a proximal end 342, a tapered distal end 344, and a central lumen 345 for slidably housing a needle shaft 324 as previously described, that allows the anchors to be smoothly and easily be pushed through the body tissue. The proximal end 342 of the anchor can be angled, e.g., by between about 15° and about 75°, between about 30° and about 60°, or about 45° in some embodiments with respect to the longitudinal axis of the anchor 320. The angled proximal end 342 can facilitate removal of the internal needle by catching on the tissues as the internal needle is retracted. The angled proximal shape also works in concert with the suture extending out hole 346 to tip the anchor when tension is applied to the suture. The anchor configuration can further be advantageous by increasing the surface area of contact to the tissues significantly increasing the pullout resistance of the anchor. In some embodiments, the anchor 320 has a first diameter D1 and a second diameter D2 at its distal end that is smaller than the first diameter D1 by at least about 10%, 20%, 30%, 40%, 50%, or more. The anchor 320 also includes a secondary lumen 346 in which a tension element such as a suture 312 can be looped through (with or without tying a knot) to secure the suture 312 to the anchor 320. In some embodiments, the anchor 320 has an axial dimension D3 of between about 4 mm and 20 mm, such as between about 8 mm and 12 mm, or about 8 mm, 10 mm, or 12 mm. In some embodiments, the anchor could have an outer diameter at the proximal end 342 or the distal end 344 of the anchor 320 of between about 0.5 mm and 5 mm, or about 2 mm.

The anchors 320 can be fully, partially, or non-bioabsorbable depending on the desired clinical result. Bioresorbable anchor components can be made, for example, of one or more of the following materials: polylactide, poly-L-lactide (PLLA), poly-D-lactide (PDLA), polyglycolide (PGA), polydioxanone, polycaprolactone, polygluconate, polylactic acid-polyethylene oxide copolymers, modified cellulose, collagen, poly(hydroxybutyrate), polyanhydride, polyphosphoester; poly(amino acids), and poly(alpha-hydroxy acid). The anchors 320 can optionally include or be fabricated from a radiopaque material or include a radiopaque marker for visualization under an imaging modality such as fluoroscopy, to confirm proper placement of the anchors 320. In some embodiments, the anchors 320 could be in a T-tag, H, or other configuration, or as disclosed, for example, in U.S. Pat. Pub. No. 2008/0009888 to Ewers et al., U.S. Pat. Pub. No. 2005/0247320 to Stack et al., or U.S. Pat. Pub. No. 2006/0025819 to Nobis et al., all of which are hereby incorporated by reference in their entireties. In some embodiments, the anchors could be coated with a drug or other biologically active material, such as a sclerosant, growth factor, or other agent capable of facilitating tissue ingrowth; an antimicrobial; or another agent depending on the desired clinical result.

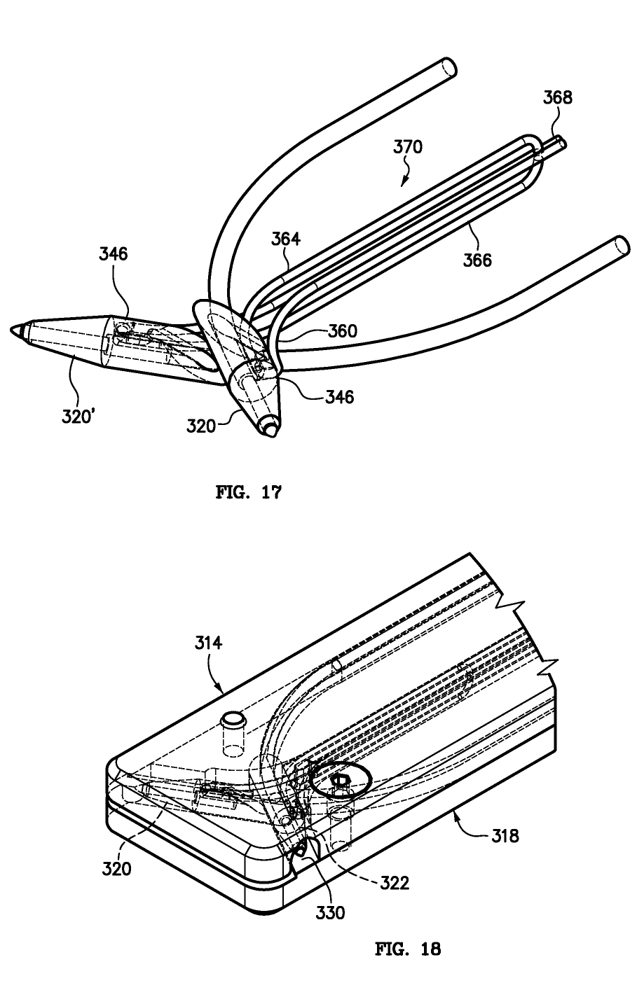

FIG. 16 schematically illustrates the distal portion 327 of the needle shaft 324 within the central lumen 342 of the anchor 320. FIG. 17 illustrates the interrelationship of a plurality of anchors 320, 320′ crossing over each other, as described and illustrated in connection with FIG. 14 above. FIG. 17 also highlights a suture loop configuration that operably connects the two anchors 320, 320′. As illustrated in one possible suture loop configuration, one suture strand 360 loops through the secondary lumen 346 of the first anchor 320 and out as segment 364, and again loops toward the second anchor 320′ as segment 366, through the secondary lumen 346 of the second anchor 320′ and then running proximally as segment 368. The loop configuration allows a single suture length to be fed through both anchors. Pulling on the middle suture loop (e.g., near 368) initially tilts the anchors 20, 320′ as described previously. After deployment of the anchors 320, 320′ through tissue the suture loop 370 can be tensioned to effectively shorten the desired muscle(s) and create a plication. FIG. 18 illustrates the anchors 320 within the pathways 330 of the housing 314 and ready for delivery as previously described. In some embodiments, one or more anchors 320 and at least a portion of attached sutures could be housed within a lumen of a delivery cannula, such as a needle or other tubular structure with a lumen therethrough, the distal end of the delivery cannula configured to pass through a tissue wall. Once the distal end of the delivery cannula penetrates the tissue wall, a stylet or other pusher member positioned within the delivery cannula lumen proximal to the anchor 320 could actuate the anchor 320 out of the distal end of the delivery cannula. The delivery cannula could then be withdrawn across the tissue wall leaving the anchor 320, and sutures could be tied together, forming a plication.

FIG. 19 illustrates an insertion tool 400, for creating a plication to draw the tissue to be plicated into a suction cavity 402 of the insertion tool 400, and assist in anchor deployment into the tissue. The suction cavity 402 includes one, two, or more suction ports 403 in communication with a source of vacuum connected to a port 404 located more proximally on the tool 400. The insertion tool 400 has a proximal end 408, a proximal handle 410, and a distal end 414. The insertion tool 400 has a lumen 409 at the proximal end 408 configured to slidably house the anchor delivery tool 300 therethrough, and coaxial or parallel to the longitudinal axis 430 of the anchor delivery tool 300. The distal end of the anchor delivery tool 300 when inserted into the insertion tool 300 enters the suction cavity 402 at port 406. In some embodiments, the insertion tool 400 has a diameter D5 of between about 15 mm and about 35 mm, such as between about 20 mm and about 30 mm, and about 25 mm.

Trans-Rectal Submucosal Plication with Anchors:

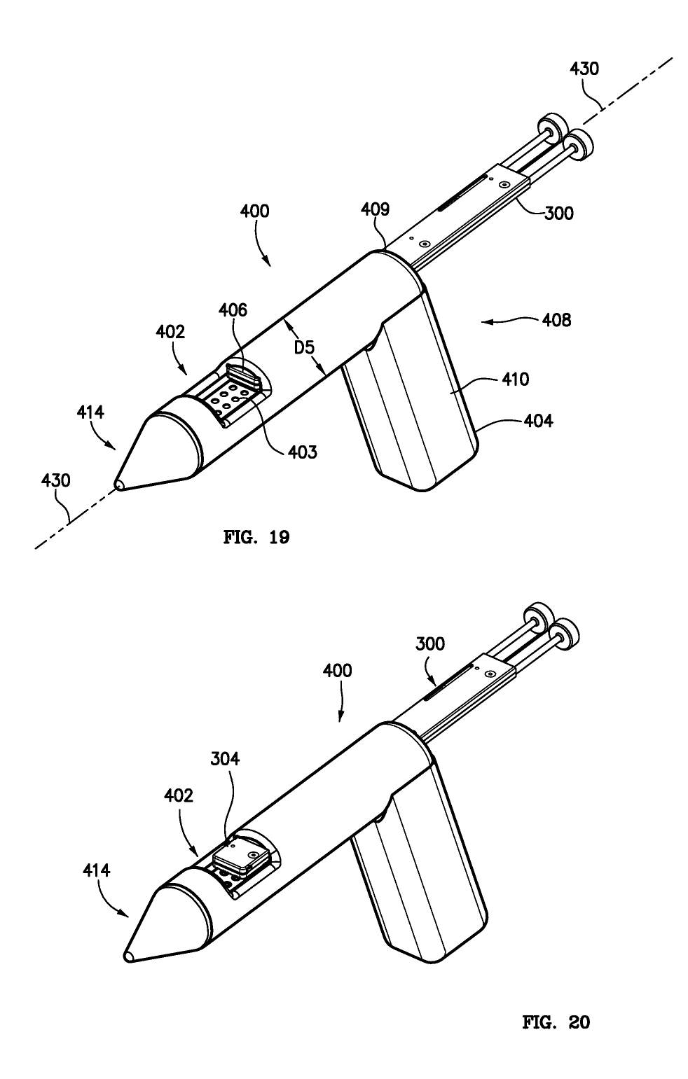

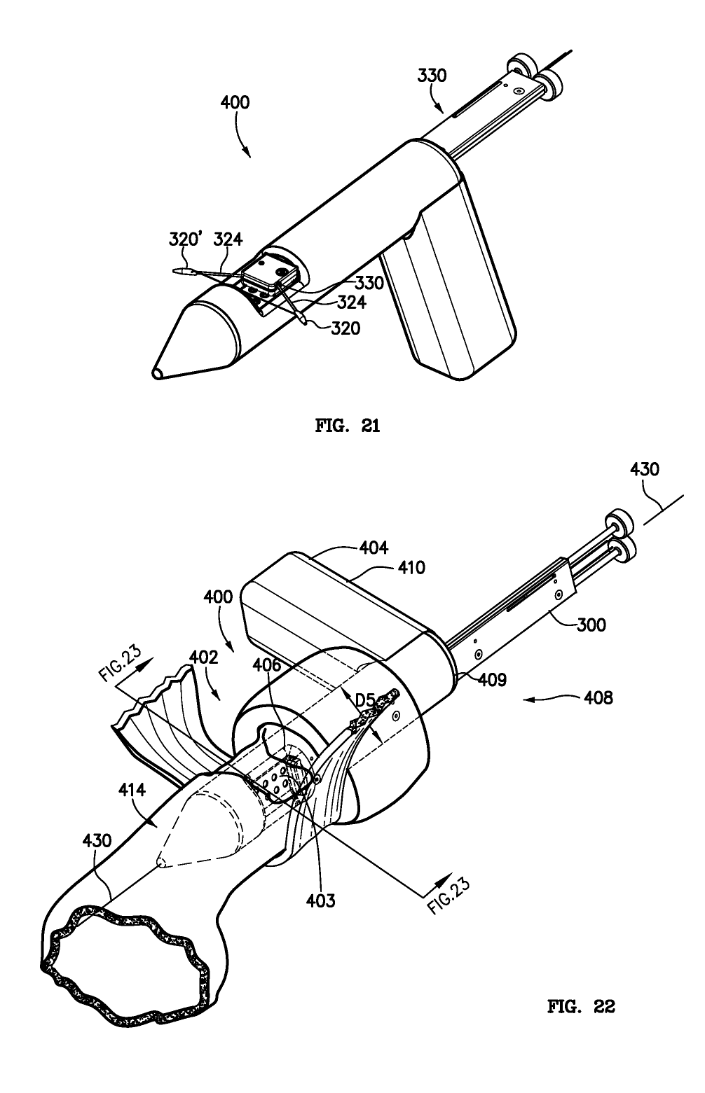

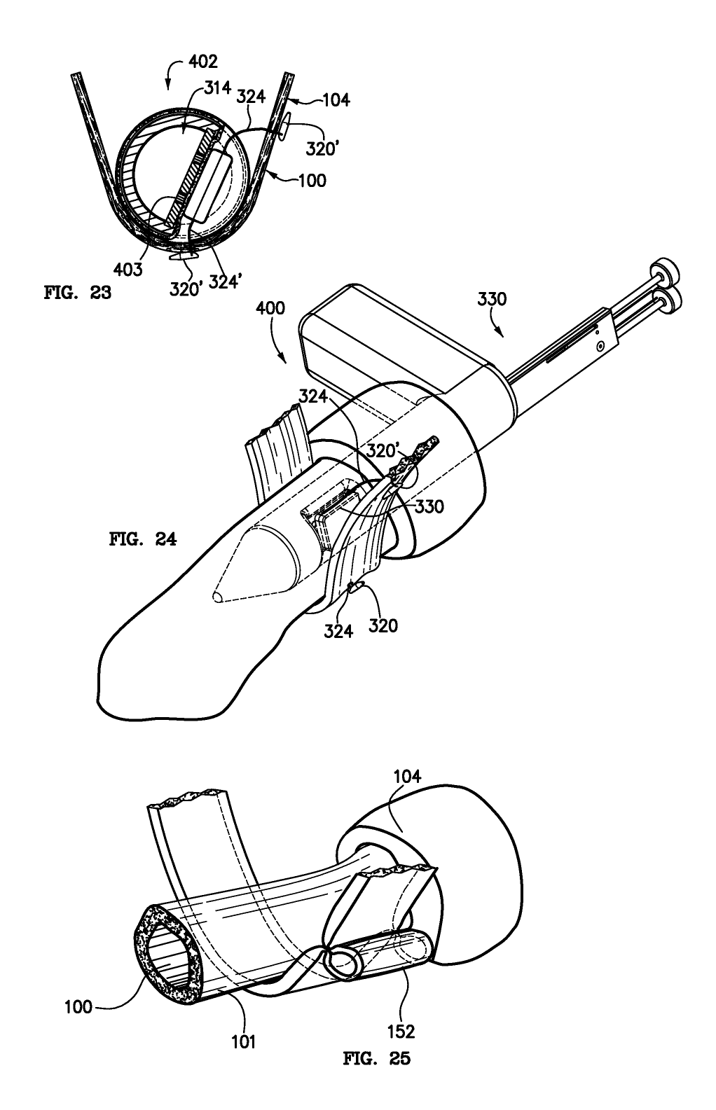

One embodiment of a method to utilize the insertion tool 400 and anchor delivery tool 300 will now be described. The distal end 414 of the insertion tool 400 and at least the suction cavity portion 402 of the insertion tool 400 can be inserted into the anal canal, and aligned and manipulated with respect to the anal canal lumen to obtain the desired suction cavity depth and orientation. In some embodiments, the insertion tool 400 is inserted between about 2 cm and 6 cm, or about 4 cm past the anal verge. Suction is then applied to pull the anal canal tissue, including the mucosa, connective tissue layers, and muscle into the suction cavity. FIG. 22 illustrates the insertion tool 400 in the anal canal with suction applied to the tissues and the distal end of the anchor delivery system. A bleb can then be formed (e.g., using a separate needle connected to a fluid, such as saline-filled syringe) to create a pocket in the submucosal, perineal or intersphincteric plane, altering the tissue geometry and assisting in formation of the plication. The distal end 304 of the anchor delivery system 300 is then advanced into the suction cavity 402, as illustrated in FIG. 20. As illustrated in FIG. 21, paired anchors 320, 320′ can then be advanced distally from the pathways 330 of the housing 314 of the anchor delivery system 300 through a tissue path created by needles 324, 324′ and deployed through one or more of the external anal sphincter, internal anal sphincter, or levator ani muscles, including the puborectalis muscle. FIG. 23 is a cross section of FIG. 22 showing the insertion tool 400 in the anal canal, the anchor delivery system 300 in the submucosal plane and two anchors 320, 320′ delivered through one or more of the external anal sphincter, internal anal sphincter, or levator ani muscles, including the puborectalis muscle 104. FIG. 24 illustrates the insertion tool 400 in the anal canal, the anchor delivery system 300 in the submucosal plane and two anchors 320, 320′ delivered through levator ani muscles, including the puborectalis muscle 104. In some embodiments, the insertion tool 400 includes stops to allow for the sequential delivery of up to 2, 4, 6, 8, 10, or more paired anchors. In some embodiments, the distance between paired anchors 320, 320′ is between about 10% and 30%, or about 20% of the circumference of the anal canal. The needles of the anchor delivery tool 300 can then be withdrawn into the housing 314, and the insertion tool 400 and the anchor delivery tool 300 withdrawn. The sutures 312 connected to the paired anchors 320 can then be tensioned, and a knot tied to secure the created plication and shorten the muscle associated with continence.

### Improved Overlapping Sphincteroplasty

Also disclosed herein is an improved wrap-around sphincteroplasty system and method. A conventional sphincteroplasty involves accessing the perineal space, visualizing and then dividing the external sphincter muscle, and mobilizing the sphincter muscle. The muscle edges are then aligned in an overlapping fashion, and mattress sutures are used to approximate the sphincter muscle. An improved procedure includes the steps of accessing the perineal space similar to the conventional procedure, and placing a plurality of bioabsorbable or non-bioabsorbable anchors connected to sutures into one or more of the internal sphincter muscle, external sphincter muscle, or puborectalis muscles. Tensioning the sutures of two anchors proximate to each other (such as separated by a distance that is, e.g., between about 10-30%, or about 20% of the circumference of the anal canal) will create an internal or external plication, which can then be secured by tying the sutures, use of staples, clips, or other attachment devices. In some embodiments, the muscle is shortened by at least about 5%, 10%, 15%, 20%, 30%, or more of its total length.

Trans-Rectal Submucosal Plication with Rotated Pins

In some embodiments, from inside the anal canal, pins can be radially expanded through the internal and external anal sphincter muscles. Rotation of the pins can create a plication in desired tissue. Further details of such a system and method are described, for example, in PCT Publication No. WO/2010/132574 to Mittal entitled “Treatment of Sphincter Dysfunction”, which is hereby incorporated by reference in its entirety.

### Trans-Vaginal Plication

Also disclosed herein is a method of plicating the puborectalis muscle, via a vaginal wall approach. An incision is created through the posterior vaginal wall, and the puborectalis muscle is visualized. Sutures, staples or the anchor deployment systems and methods disclosed herein can be utilized to plicate the puborectalis muscle. The plication can be formed facing either internally or externally with respect to the vaginal wall and can be on one or both lateral aspects of the puborectalis muscle.

Posterior-Lateral Transrectal Submucosal Plication with Anchors

Another method involves plication of one, two or more of the internal anal sphincter muscle, external anal sphincter muscle, and the puborectalis muscle by creating a 4-5 cm deep dissection plane superficial to (radially outward of) the external anal sphincter muscle and the puborectalis muscle at the 4-5 o-clock and/or 7-8 o-clock positions with respect to the anal canal with the patient positioned on their back. Then, a submucosal pocket or bleb can be created in the anal canal on the deep (radially inward) side of the muscle. Using the stapling or anchoring devices disclosed herein, the aforementioned internal anal sphincter muscle, external anal sphincter muscle, and the puborectalis muscle can all be plicated. As disclosed above, the plication may face internally or externally with respect to the anal canal lumen. Alternatively, a pocket is created at the 4-5 o-clock and/or 7-8 o-clock positions with respect to the anal canal with the patient positioned dorsally in the intersphincteric plane between the inner anal sphincter and the external anal sphincter and puborectalis muscles. Using the stapling or anchoring devices disclosed herein, the aforementioned, external anal sphincter muscle and/or the puborectalis muscle can be plicated. As disclosed above, the plication could face internally or externally with respect to the anal canal. FIG. 25 is a schematic view of the anal canal 100 showing an external plication 153 of the internal and external anal sphincters using anchors 320 as previously described. In some embodiments, in addition to or in lieu of anchors 320, sutures such as purse-string type sutures can be utilized, and tied manually or with another suture-passing device.

### Muscle Shortening Using Delivered Energy

In another embodiment, a muscle associated with continence, such as the puborectalis muscle, is shortened by creating scar tissue along a portion of the length of the muscle. The muscle of interest (e.g., the puborectalis muscle, internal, and/or external anal sphincter) can be accessed transanally, transvaginally or through a dissection plane as described elsewhere herein. Next, a source of energy, a chemical, a mechanical device, or a combination of the foregoing can be utilized to create scar tissue and/or plicate the muscle, effectively shortening the muscle and increasing muscle tone. Possible energy sources that can shorten muscle tissue include laser, ultrasound such as focused ultrasound or high intensity focused ultrasound (HIFU), microwave, infrared, visible, or ultraviolet light energy, electric field energy, magnetic field energy, cryoablation, combinations of the foregoing, or other modalities. Mechanical devices for shortening or plicating muscles could include tension elements such as sutures, clips, bands, staples, or anchors as described elsewhere herein. Chemical agents could include to create scar tissue and/or form a plication include adhesives such as cyanoacrylate, e.g., 2-octyl cyanoacrylate, and/or a sclerosing agent such as hypertonic saline, sodium tetradecyl sulfate, chromated glycerol, tetracycline, talc, bleomycin, or polydocanol. In some embodiments, a cyanoacrylate can be an aliphatic 2-cyanoacrylate ester such as an alkyl, cycloalkyl, alkenyl or alkoxyalkyl 2-cyanoacrylate ester. Other adhesives that can be used include a biological glue such as a bovine serum albumin-gluteraldehyde combination (e.g., BIOGLUE, Cryolife, Atlanta, Ga.), PVA, Biogard, collagen, fibrinogen, fibronectin, vitronectin, laminin, thrombin, gelatin, mixtures thereof, or other biocompatible adhesives. Botulinum toxin and peroxides are other non-limiting examples of possible chemicals that can be utilized.

Although certain embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. In addition, while a number of variations have been shown and described in varying detail, other modifications, which are within the scope of the present disclosure, will be readily apparent to those of skill in the art based upon this disclosure. It is also contemplated that various combinations or subcombinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described above. For all of the embodiments described above, the steps of any methods need not be performed sequentially.

We claim:

1. A method of performing a sphincter repair in a patient, the method comprising the steps of:
inserting a stabilization and an anchor deployment tool into the patient; the anchor deployment tool housing a plurality of tissue anchors connected to tension elements housed within the anchor deployment tool;
positioning the stabilization tool to access a muscle associated with a sphincter;
stabilizing tissue with the stabilization tool, the tissue including or in communication with the muscles associated with the sphincter;
advancing the anchor deployment tool into the tissue cavity;
advancing the plurality of tissue anchors out of the anchor deployment tool and transmurally through the tissue;
tensioning the anchors to shorten the muscle associated with the sphincter; and
withdrawing the stabilization tool from the patient.

2. The method of claim 1, wherein inserting a stabilization tool comprises inserting the stabilization tool into the anal canal.

3. The method of claim 1, wherein inserting a stabilization tool comprises inserting the stabilization tool into the vagina.

4. The method of claim 1, wherein inserting a stabilization tool comprises inserting the stabilization tool percutaneously into the perineal space.

5. The method of claim 1, wherein the muscle comprises one or more of the group consisting of the internal anal sphincter, the external anal sphincter, and the levator ani including the puborectalis muscle.

6. The method of claim 1, wherein the anchors comprise a proximal end, a distal end, an elongate body, and a central lumen.

7. The method of claim 6, wherein the central lumen has a first inner diameter at the distal end that is at least 10% smaller than a second inner diameter at the proximal end.

8. The method of claim 6, wherein the proximal end has a surface that is angled by between about 5 degrees and about 75 degrees with respect to a longitudinal axis of the anchor.

9. The method of claim 6, wherein the anchor has an axial length of between about 4 mm and about 20 mm.

10. The method of claim 1, wherein advancing the plurality of tissue anchors out of the anchor deployment tool comprises advancing the tissue anchors at an angle of between about 15 degrees and about 75 degrees from a longitudinal axis of the anchor deployment tool.

11. The method of claim 1, wherein the sphincter comprises the anal sphincter.

12. The method of claim 1, wherein tensioning the anchors comprises applying a force to the tension elements connected to the anchors.

13. A method of treating fecal incontinence in a patient, the method comprising the steps of:
inserting a tool to stabilize the tissue and an anchor deployment tool into the patient; the anchor deployment tool housing a plurality of tissue anchors connected to tension elements housed within anchor deployment tool into a patient;
positioning the stabilization tool to access at least one muscle selected from the group consisting of the internal anal sphincter muscle, the external anal sphincter muscle, and the puborectalis muscle;
stabilizing tissue with the stabilization tool to create a tissue fold, the tissue in communication with or comprising the sphincter muscle(s);
creating a pocket within the tissue space;
advancing the anchor deployment tool into the cavity;
advancing the plurality of tissue anchors out of the anchor deployment tool and transmurally through the tissue;
tensioning the anchors to shorten the muscle associated with the sphincter; and
withdrawing the stabilization tool from the patient.
